(19) United States
(12) Patent Application Publication
(10) Pub. No.: US 2011/0015470 A1
(43) Pub. Date: Jan. 20, 2011

(54) STROBE LIGHT FOR BRAIN WAVE ENTRAINMENT

(76) Inventors: Martin L. Lenhardt, Hayes, VA (US); Raymond Gargiulo, Richmond, VA (US); Robert Guinta, Woodcliff Lake, NJ (US)

Correspondence Address: Hershkovitz & Associates, LLC, 2845 Duke Street, Alexandria, VA, 22314, US

(21) Appl. No.: 12/811,473

(22) PCT Filed: Jan. 5, 2009
(86) PCT No.: PCT/US09/30089

§ 371 Date: Oct. 4, 2010

Related U.S. Application Data
(60) Provisional application No. 61/019,008, filed on Jan. 4, 2008.

(51) Int. Cl. A61M 21/02

(52) U.S. Cl. 600/27

(57) ABSTRACT

A strobe device is used to affect brain wave patterns, such as for personal defense by causing disorientation, or for therapeutic use by causing calming.

### BACKGROUND OF THE INVENTION

1. Field of Invention

The present invention relates to device for affecting brain wave patterns.

2. Background of the Invention

The brain's neuronal firing patterns can be affected by a number of means. For example, excitement, sleep deprivation, drug-usage, and simply day-to-day activities, such as listening to music or speaking, produce shifts in the manner and intensity of the brain's neuronal firing patterns. To the extent that such firing patterns can be induced, there exists great potential for modification of behavior, physiological state, and other body status characteristics. These patterns are typically referred to as brain waves and comprise a number of frequencies as measured by an electroencephalogram, including: Delta waves (up to 3 Hz) which is normally seen in slow wave sleep; Theta waves (4 to 7 Hz) which typically indicates drowsiness or arousal; Alpha waves (8 to 12 Hz but sometimes slower) which indicate an awake but relaxed state; Beta waves (12 to about 30 Hz) which correlate with concentrated thinking; and Gamma waves (26 to 100 Hz). Accordingly, there is a present need for a device which can advantageously induce one or more of these or other brain wave states.

### SUMMARY OF INVENTION

The present invention is a high energy light source that pulses at frequencies typical of brain wave frequencies as measured in clinical electroencephalography. Pulsing in the “Alpha range” can disorientate and pulsing in other ranges can calm. In a second embodiment the invention is packaged as a cell phone with a preset pulser frequency for use as a personal safety device.

The present invention is, in one or more embodiments, a device for affecting brain wave patterns comprising at least one light emitting diode (LED) adapted to produce a patterned output of light, at least one lens adapted to focus the output of the LED, a frequency selection switch adapted to adjust the rate of the patterned output of light of said LED; and an energy selection switch adapted to adjust the intensity of the patterned output of light of said LED. The device may be portable and may further comprise a flicker switch for fine-tuning the frequency and/or pattern of the patterned output of light of said LED.

The present invention is also, in one or more embodiments, a method of using the above device to disorient a person comprising powering the device and pointing the patterned output of light produced by the LED toward said person; and a method of using the above device to calm a person comprising powering the device and pointing the patterned output of light produced by the LED toward said person.

### BRIEF DESCRIPTION OF THE DRAWINGS

So that the manner in which the above-recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a circuit diagram for a device that provides a strobe light pattern causing disorientation or calming.

### DETAILED DESCRIPTION OF THE INVENTION

An external stimulus to the senses, can, if properly patterned, interact with the electrical firing of neurons within the brain. For example, it is well known that certain strobe lights can cause epileptics to have seizures. The present invention, in one or more embodiments, utilizes a hand-held strobe for the purposes of affecting the firing patterns of the brain. In particular, a strobe pattern is described which coincides with alpha brain rhythms to disorient persons. Such an application would have numerous applications, including self-defense and law enforcement applications. The device gives individuals additional time to react or escape from a threat. In clinical observations, Dr. Raymond Gargiulo, has observed that strobe pulsing is effective to disorient individuals. Lower pulsing rates may be used to instill a sense of calm in a person and therefore the invention also has therapeutic uses. neurons that produce the bioelectricity recorded in the hospital. The invention may comprise the following elements:
1) A lens of a multiple LED type with a spectral range of 0 to about 100 or more meters: The lens is adapted to allow for short and long distance use with equal efficacy. The preferred embodiment is non-contact in the under 10 meter range. It is contemplated that other high energy beams can be used;
2) Light Emitting Diodes
3) Frequency Selection Switch: A switch assembly which allows pulsation with light at selected frequencies from under 1 Hertz to about 50 Hertz. The switch assembly may be adapted to allow manual adjustment of the frequency pulse within that range;
4) A Power Switch to turn the device on and off;
5) An Energy Level Switch: A switch to allow daytime or night-time luminance;
6) (Optional) A Flicker Pulsation Adjustment: An adjustment to allow fine tuning of the pulsing rate.

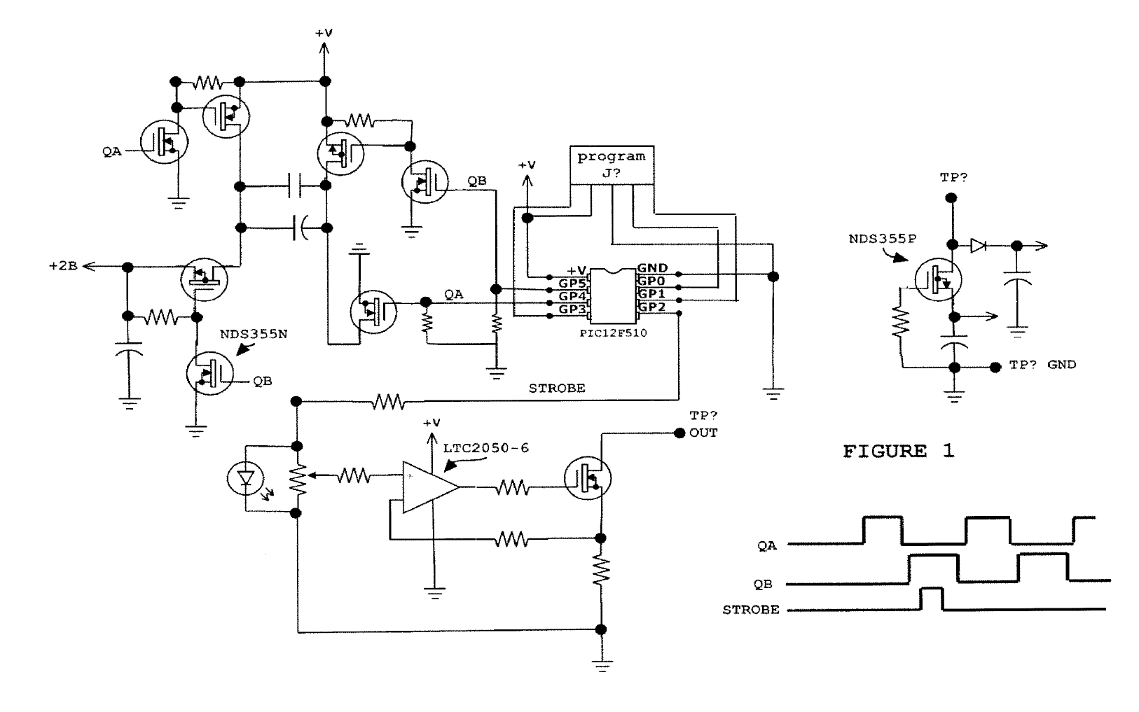

An exemplary embodiment of the current device is shown in FIG. 1, which indicates a possible circuit board arrangement for affecting a strobe pattern according to the present invention.

The LEDs may be arranged in an array, such as 3×3, 4×4, 2×3, or others, although in an exameplary embodiment, the LEDs are in a 3×5 pattern. The LEDs have a lens placed in a position adapted to provide maximal transmission of the light bursts. Such an arrangement may be installed on the back of a cell-phone or other personal items for use as a personal protection device.

The device may also function to induce brain-wave patterns by the use of pulsing patterns.

In the foregoing description, certain terms and visual depictions are used to illustrate the preferred embodiment. However, no unnecessary limitations are to be construed by the terms used or illustrations depicted, beyond what is shown in the prior art, since the terms and illustrations are exemplary only, and are not meant to limit the scope of the present invention. It is further known that other modifications may be made to the present invention, without departing the scope of the invention, as noted in the appended claims.

I claim:

1) A method for affecting brain wave patterns comprising the steps of
i. using at least one light emitting diode (LED) adapted to produce a patterned output of light,
ii. using at least one lens adapted to focus the output of the LED,
iii. using a frequency selection switch adapted to adjust the rate of the patterned output of light of said LED; and
iv. using an energy selection switch adapted to adjust the intensity of the patterned output of light of said LED.

2) The method of claim 1 in which said method further comprises using a flicker switch to fine-tune the frequency and/or pattern of the patterned output of light of said LED.

3) The method of claim 1 in which said method is used to disorient a person comprising the additional steps of powering the device and pointing the patterned output of light produced by the LED toward said person.

4) The method of claim 1 in which said method is used to calm a person comprising the additional steps of powering the device and pointing the patterned output of light produced by the LED toward said person.
